(19) United States
(12) Patent Application Publication
(10) Pub. No.: US 2016/0338941 A1
(43) Pub. Date: Nov. 24, 2016

(54) SYSTEMS, DEVICES AND METHODS FOR APPLYING SUBSTANCES TO SKIN

(71) Applicants: David Reece DeMille, Cedar Hills, UT (US); Hiram James Thompson, Cedar Hills, ID (US); Douglas Michael Gilmore, Cedar Hills, ID (US)

(72) Inventors: David Reece DeMille, Cedar Hills, UT (US); Hiram James Thompson, Cedar Hills, ID (US); Douglas Michael Gilmore, Cedar Hills, ID (US)

(21) Appl. No.: 15/157,411

(22) Filed: May 18, 2016

Related U.S. Application Data
(60) Provisional application No. 62/162,917, filed on May 18, 2015.

(51) Int. Cl. A61K 8/92; A61K 9/70; A61Q 19/00

(52) U.S. Cl. CPC A61K 8/922; A61Q 19/00; A61K 9/70

(57) ABSTRACT

Devises, methods and systems for applying substances to a user's skin are disclosure. A device for applying at least one Essential Oil to a user's skin may include a first member, a second member, and at least one third member. The disclosure may allow users to customize the amount, type, mixture and level of concentration of Essential Oils used.

### CROSS-REFERENCE TO RELATED APPLICATIONS

The present patent document claims the benefit of and priority to the filing date under 35 U.S.C. §119(e) of Provisional U.S. Patent Application Ser. No. 62/162,917, entitled “SYSTEMS, DEVICES AND METHODS FOR APPLYING SUBSTANCES TO SKIN” and filed on 18-May-2015, the entire contents of which is hereby incorporated by reference as part of this application.

### BACKGROUND

1. Technical field

The disclosure relates at least to systems, devices and methods for applying substances to skin.

2. Discussion of related field

People may use various Essential Oils or other forms of liquids, gels, oils and/or other substances for medicinal and/or therapeutic purposes, including relieving aches, plains, swelling, weakness, discomfort, etc. Essential Oils may be administered in various ways, one of which is by placing a patch containing Essential Oils onto a person's skin. Current Essential Oil patches, however, may be pre-oiled at the manufacturing stage and are not customizable. That is, current Essential Oil patches do not allow users to customize the amount, type, mixture and level of concentration of substances used.

If users had the ability to customize the amount, type, mixture and level of concentration of substances used, such may provide users various benefits, including, for example, the following: greater options as certain substances may not be in pre-oiled patches; the ability to determine the level of concentration of the desired substances and thereby increase or decrease the level of potency of the substances; and the ability to mix multiple substances together for a variety of purposes, such as, for example, to masks the smell of a substance and/or combine medical purposes associated with two or more substances.

In light of the foregoing discussion, there may be at least a need to provide patches or other forms of materials that may be designed to receive Essential Oils and/or other substances from users in amounts, types, mixtures and concentration of their choosing and to be placed on their bodies to allow the substances to directly contact users' skin.

### SUMMARY

In one aspect, a device for applying at least one Essential Oil to a user's skin, may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member is made from material that absorbs Essential Oils and communicates the at least one Essential Oil onto a user's skin when at least an aspect of the at least one third member is in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; and wherein the first perimeter may be operably connected to the second perimeter such that said connection between the first perimeter and the second perimeter substantially remains in place when a user applies the at least one Essential Oil to the device and when the user removes the device from the user's skin.

Implementations may include one or more of the following features. One side of the at least one third member may be surrounded by the first perimeter and an alternative side of the at least one third member may be surrounded by the second perimeter. The first member and the second member may each substantially circular in shape. Multiple devices may be releasably and operably connected to a sheet which may include a sheet bottom surface, a sheet top surface and at least one sheet opening, wherein at least a portion of at least one third member may be accessible through the at least one sheet opening such that the at least one Essential Oil may be applied to the devices releasably and operably connected to the sheet. The first member and the second member may each be substantially rectangular in shape. Multiple devices may be operably connected together. Multiple devices may be operably connected together to form a band wherein multiple devices may be operably connected end to end and capable of being rolled together. The second member which may include kinesiology tape. The device may include: a cover member may include a cover bottom surface, a cover top surface, a cover perimeter defined by the entire outer boundary of cover member, and at least one cover opening; wherein cover member may be configured to releasably cover and surround at least some aspect of the first bottom surface for the purpose of covering and protecting at least some aspect of the first bottom surface until a user removes the cover member from the first bottom surface; and wherein at least a portion of at least one third member may be accessible through the at least one cover opening.

In one aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; and wherein the first perimeter may be operably connected to the second perimeter such that said connection between the first perimeter and the second perimeter substantially remains in place when a user applies the at least one Essential Oil to the device and when the user removes the device from the user's skin; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and wherein one side of the at least one third member may be surrounded by the first perimeter and an alternative side of the at least one third member may be surrounded by the second perimeter; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and wherein the first member and the second member may be each substantially circular in shape; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and wherein multiple devices may be releasably and operably connected to a sheet which may include a sheet bottom surface, a sheet top surface and at least one sheet opening, wherein at least a portion of at least one third member may be accessible through the at least one sheet opening such that the at least one Essential Oil may be applied to the devices releasably and operably connected to the sheet; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and wherein the first member and the second member may be each substantially rectangular in shape; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and wherein multiple devices may be operably connected together; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and wherein multiple devices may be operably connected together to form a band wherein multiple devices may be operably connected end to end and capable of being rolled together; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and wherein the second member may be made from kinesiology tape; placing the device containing the at least one Essential Oil directly onto a target area of a user's skin for a desired length of time; and removing the device after the desired length of time has passed.

In another aspect a method for applying at least one Essential Oil to a user's skin, may include: applying at least one Essential Oil onto a device which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; wherein the first perimeter may be operably connected to the second perimeter such that said connection between the first perimeter and the second perimeter may substantially remain in place when a user applies the at least one Essential Oil to the device and when the user removes the device from the user's skin; and a cover member which may include a cover bottom surface, a cover top surface, a cover perimeter defined by the entire outer boundary of cover member, and at least one cover opening; wherein cover member may be configured to releasably cover and surround at least some aspect of the first bottom surface for the purpose of covering and protecting at least some aspect of the first bottom surface until a user removes the cover member from the first bottom surface; and wherein at least a portion of at least one third member may be accessible through the at least one cover opening; placing the device containing the at least one Essential Oil directly onto a target area of a user's skin for a desired length of time; and removing the device after the desired length of time has passed.

In another aspect a system for applying at least one Essential Oil to a user's skin, may include: a device for applying at least one Essential Oil to a user's skin which may include: a first member which may include a first bottom surface, a first top surface, a first perimeter defined by the entire outer boundary of the first member, and at least one opening; a second member which may include a second bottom surface, a second top surface, and a second perimeter defined by the entire outer boundary of the second member; at least one third member which may include at least one third bottom surface, at least one third top surface, and at least one third perimeter defined by the entire outer boundary of the at least one third member; wherein the at least one third member may be situated in between the second member and the first member; wherein a user may access the at least one third member through the at least one opening of the first member; wherein the at least one third member may be made from material that absorbs Essential Oils and communicates the at least one Essential Oil onto a user's skin when at least an aspect of the at least one third member may be in contact with a user's skin through the at least one opening of the first member; wherein the first bottom surface which may include material possessing non-porous qualities in order to aid in the seepage of the at least one Essential Oil onto a user's skin from the at least one third member through the at least one opening of the first member when the at least one Essential Oil may be applied to the at least one third member; wherein the first bottom surface which may include material possessing adhesive qualities in order to stick to a user's skin; wherein the second top surface which may include material possessing non-porous qualities in order to prevent seepage of the at least one Essential Oil from the second top surface when the at least one Essential Oil may be applied to the at least one third member; and wherein the first perimeter may be operably connected to the second perimeter such that said connection between the first perimeter and the second perimeter may substantially remain in place when a user applies the at least one Essential Oil to the device and when the user removes the device from the user's skin; at least one Essential Oil; and at least one user which may include at least one area of skin.

These general and specific aspects may be implemented by using systems, apparatuses, devices, means, methods and structures and/or any combination thereof

Certain implementations may provide one or more of the following advantages. Embodiments may not achieve any or all of the listed advantages. Further, this is not an exhaustive list of all possible advantages of the disclosure. One or more embodiments of the disclosure may be configured to be and/or provide users the following.

In some embodiments, the disclosure may provide users the ability to customize the amount, type, mixture and level of concentration of substances used.

In some embodiments, the disclosure may provide users greater options compared to the current art as certain substances may not be in existing pre-oiled patches.

In some embodiments, the disclosure may provide users the ability to determine the level of concentration of the desired substances and thereby increase or decrease the level of potency of the substances.

In some embodiments, the disclosure may provide users the ability to mix multiple substances together for a variety of purposes, such as, for example, to masks the smell of a substance and/or combine medical purposes associated with two or more substances.

In some embodiments, the disclosure may provide users not only the benefit of using certain substances (e.g. Essential Oils), but also the benefits associated with whatever medical tape or other materials used in conjunction with the disclosure and said substances.

In some embodiments, the disclosure may provide users the ability to cover and treat larger target areas.

Other aspects and advantages may be apparent from the following detailed description, the accompanying drawings, and/or the claims.

### BRIEF DESCRIPTION OF THE DRAWINGS

Various embodiments of the disclosure will now be discussed with reference to the appended drawings. It is appreciated that these drawings depict only typical embodiments of the disclosure and are not to be considered limiting of its scope.

FIG. 1 is a perspective top view of one embodiment of a device showing three components of the device.

FIG. 2 is a perspective top view of one embodiment of one component of the device illustrated in FIG. 1.

FIG. 3 is a bottom view of one embodiment of the component illustrated in FIG. 2.

FIG. 4 is a perspective top view of one embodiment of another component of the device illustrated in FIG. 1.

FIG. 5 is a bottom view of one embodiment of the component illustrated in FIG. 4.

FIG. 6 is a perspective top view of one embodiment of another component of the device illustrated in FIG. 1.

FIG. 7 is a bottom view of one embodiment of the component illustrated in FIG. 6.

FIG. 8 is a perspective top view of one embodiment of the components of the device illustrated in FIG. 1 configured together.

FIG. 9 is a perspective bottom view of one embodiment of the components of the device illustrated in FIG. 1 configured together.

FIG. 10 is a bottom view of one embodiment of the components of the device illustrated in FIG. 1 configured together.

FIG. 11A is a perspective top view of one embodiment of a device showing four components of the device.

FIG. 11B is a perspective top view of one embodiment of one component of the device illustrated in FIG. 11A.

FIG. 11C is a bottom view of one embodiment of the components of the device illustrated in FIG. 11A configured together.

FIG. 12A is a view of one embodiment of multiple devices and some of their components configured to one embodiment of a sheet.

FIG. 12B is an alternative view of the devices and sheet illustrated in FIG. 12A.

FIG. 13 is a perspective top view of one embodiment of a device showing three components of the device, albeit in a different configuration as compared to FIG. 1.

FIG. 14 is a perspective top view of one embodiment of one component of the device illustrated in FIG. 13.

FIG. 15 is a perspective top view of one embodiment of another component of the device illustrated in FIG. 13.

FIG. 16 is a perspective top view of one embodiment of another component of the device illustrated in FIG. 13.

FIG. 17 is a perspective top view of one embodiment of the components of the device illustrated in FIG. 13 configured together.

FIG. 18A is a perspective bottom view of one embodiment of the components of the device illustrated in FIG. 13 configured together.

FIG. 18B is a bottom view of one embodiment of the components of the device illustrated in FIG. 13 configured together.

FIG. 19A is a perspective top view of one embodiment of a device showing four components of the device, albeit in a different configuration as compared to FIG. 11A.

FIG. 19B is a bottom view of one embodiment of the components of the device illustrated in FIG. 19A configured together.

FIG. 20A is a top view of one embodiment of multiple devices and some of their components configured to a sheet, albeit in a different configuration as compared to FIG. 12A.

FIG. 20B is a bottom view of one embodiment of the multiple devices and sheet illustrated in FIG. 20A.

FIG. 21A is a top view of one embodiment of multiple devices configured together.

FIG. 21B is a perspective view of one embodiment of multiple devices configured together in a roll like configuration.

FIG. 21C is a perspective view of one embodiment of multiple devices configured together in a roll like configuration, albeit in a different configuration as compared to FIG. 21B.

FIG. 22 is a CAD drawing illustrating a perspective bottom view of one embodiment of a device showing three components of the device.

FIG. 23A is a CAD drawing illustrating a bottom view of one embodiment of the components of the device illustrated in FIG. 22 configured together.

FIG. 23B is a CAD drawing illustrating a top view of one embodiment of the components of the device illustrated in FIG. 22 configured together.

FIG. 24 is a CAD drawing illustrating a top view of one embodiment of multiple devices and some of their components configured to a sheet.

FIG. 25 is a CAD drawing illustrating a bottom view of one embodiment of the multiple devices and sheet illustrated in FIG. 24.

FIG. 26 is a CAD drawing illustrating a bottom view of one embodiment of components of a device.

FIG. 27 is a CAD drawing illustrating a top view of one embodiment of components of a device to be configured to the components illustrated in FIG. 26.

FIG. 28 is a CAD drawing illustrating a bottom view of one embodiment of components of a device, some of which are illustrated in FIGS. 26 and 27.

FIG. 29 is a bottom view of one embodiment of components of a device.

FIG. 30 is a top view of one component of a device as illustrated in FIG. 29.

FIG. 31 is a bottom view of one embodiment of components of a device, some of which are illustrated in FIGS. 29 and 30.

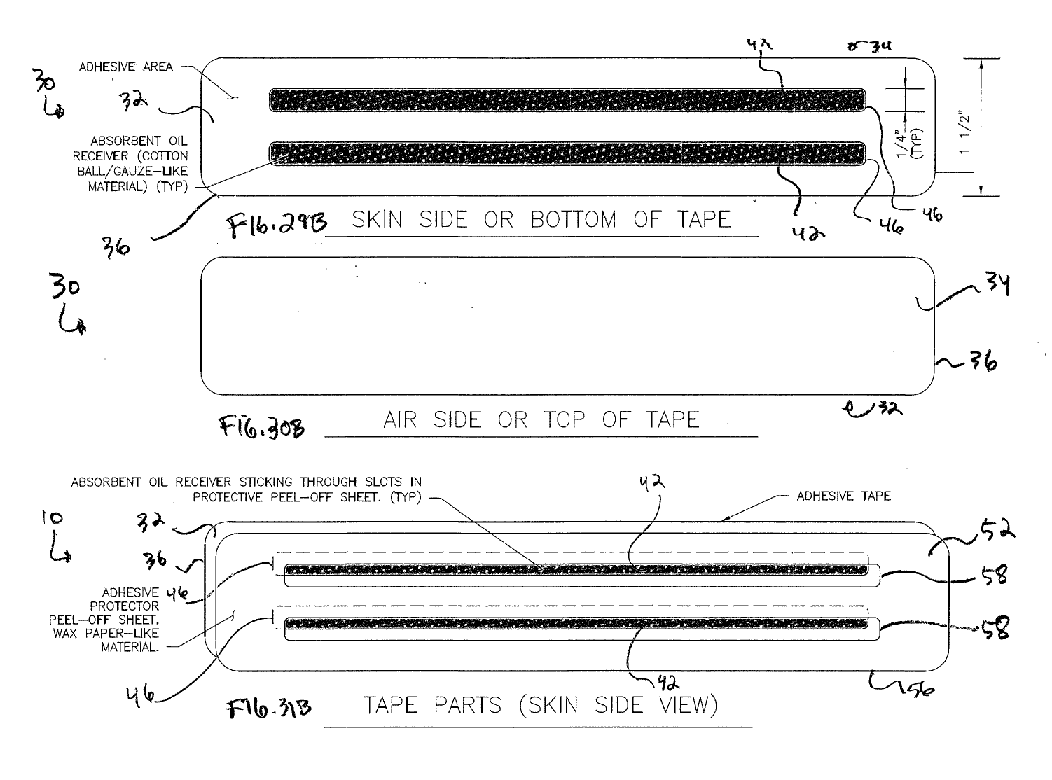

FIG. 29B is a CAD drawing illustrating bottom view of one embodiment of components of a device.

FIG. 30B is a CAD drawing illustrating a top view of one component of a device as illustrated in FIG. 29B.

FIG. 31B is a CAD drawing illustrating a bottom view of one embodiment of components of a device, some of which are illustrated in FIGS. 29B and 30B.

### DETAILED DESCRIPTION

The following description illustrates principles of the disclosure which may be applied in various ways to provide different embodiments. There may be many different forms of embodiments of the disclosure, and as such, embodiments should not be limited to those set forth herein and shown in the accompanying drawings. That which is set forth herein and shown in the accompanying drawings is offered to illustrate the principles of the disclosure and one or more embodiments, and not as limitations. Other variations of the disclosure may be included within the principles of the disclosure. The disclosure may be configurable, adaptable and customizable to meet the various needs of various users in various circumstances.

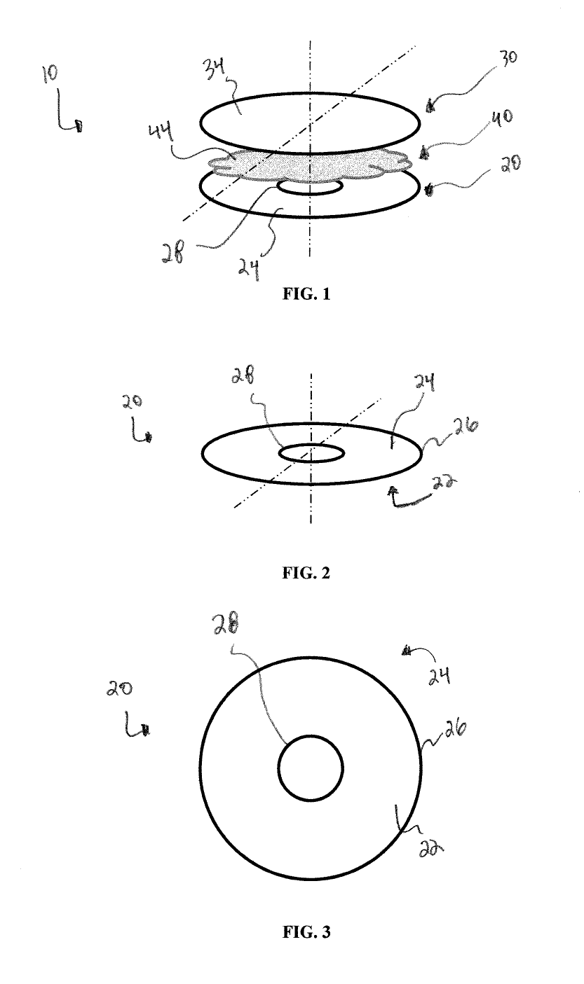

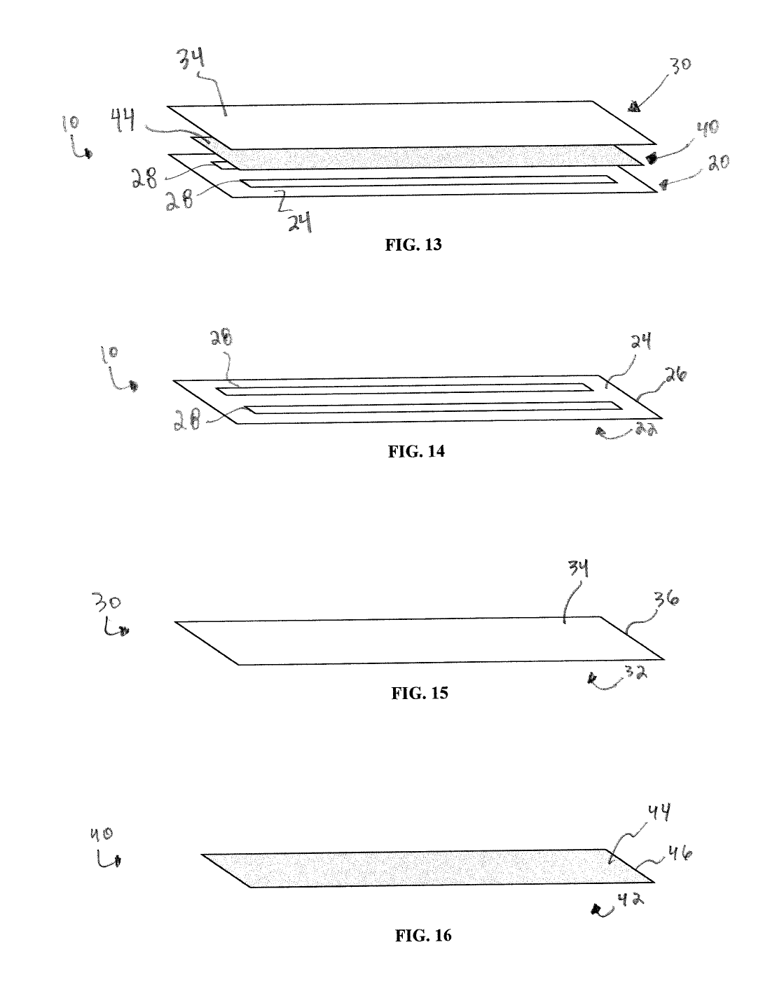

FIGS. 1 and 13 (and elsewhere) show embodiments of certain components of a device 10 for applying substances to a user's skin. In one or more embodiments, the substances that device 10 may apply may vary, including, for example, oils, liquids, and/or gels of any type or kind, including Essential Oils. In one or more embodiments, the surfaces upon which the substances may be applied may vary, including, for example, arms, necks, backs, stomachs and/or any other surface of the human body. In one or more embodiments, a user may use device 10 to apply various substances to various surfaces for various reasons, such as, for example, experimental, medicinal and/or therapeutic purposes, including, for example, to relieve aches, plains, swelling, weakness, discomfort, etc. In one or more embodiments, some or all of the components of device 10 may be manufactured from and/or by various materials and/or means and/or combinations of the same. In one or more embodiments, some or all of the components of device 10 may be configured to a variety of positions, sizes, shapes, dimensions, textures, colors, smells, orders, characteristics, and/or designs.

Referring to FIGS. 1 and 13 (and elsewhere), in one or more embodiments, device 10 may include a first member 20, a second member 30, and at least one third member 40. In one or more embodiments, at least one third member 40 may be situated between first member 20 and second member 30.

FIGS. 2, 3, and 14 (and elsewhere) show embodiments of the first member 20 which may include a first bottom surface 22, a first top surface 24, a first perimeter 26, and at least one opening 28. The first perimeter 26 is defined by the entire outer boundary of first member 20. The size of the at least one opening 28 may vary. As stated above with regards to each component of device 10, first member 20 and each of its parts may be configured to a variety of positions, sizes, shapes, dimensions, textures, colors, smells, orders, characteristics, and/or designs (including those not illustrated in FIGS. 2, 3 and 14).

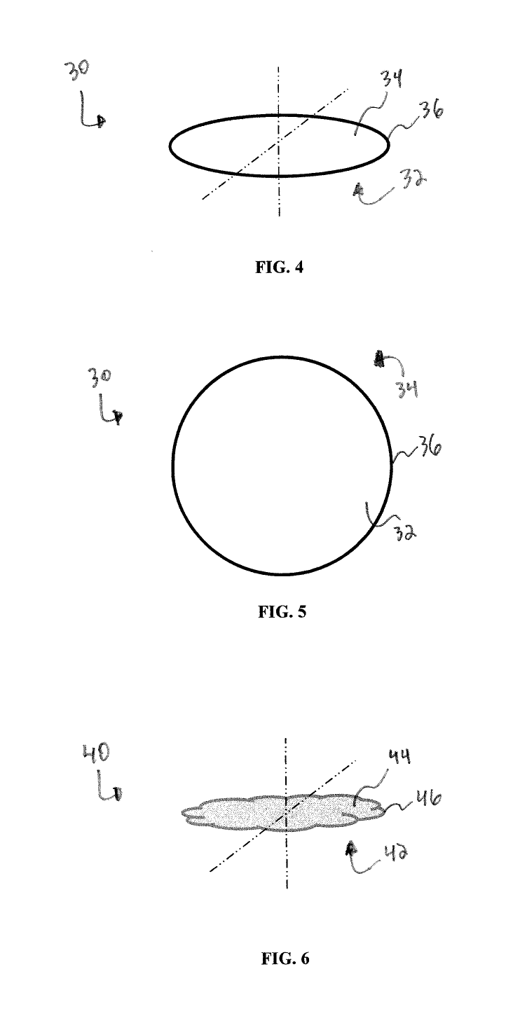

FIGS. 4, 5 and 15 (and elsewhere) show embodiments of the second member 30 comprising a second bottom surface 32, a second top surface 34, and a second perimeter 36. The second perimeter 36 is defined by the entire outer boundary of second member 30. As stated above with regards to each component of device 10, second member 30 and each of its parts may be configured to a variety of positions, sizes, shapes, dimensions, textures, colors, smells, orders, characteristics, and/or designs (including those not illustrated in FIGS. 4, 5 and 15).

In one or more embodiments, first member 20 and second member 30, and each of their parts, may be configured from various materials and/or combinations of materials, including, for example, cotton, plastic, synthetic blend, cloth, kling, kerlix, bamboo, elastic, rubber, hemp, petroleum based, woven, polyester, latex, wool, paper, knit, silk, flannel, muslin, gauze and the like and/or other materials. In one or more embodiments, the materials and/or combinations of materials from which first member 20 and second member 30, and each of their parts, may be configured from and/or may include materials possessing porous and/or non-porous qualities and/or adhesive and/or non-adhesive qualities (such as, for example, patches, tapes (e.g. kinesiology tape), Band-Aids, and other medical adhesive materials and the like and/or other materials). For example, first bottom surface 22 may be configured from material that may adhere to a user's skin and be non-porous in nature and second top surface 34 may be configured from material that may not adhere to a user's skin but may be non-porous in nature.

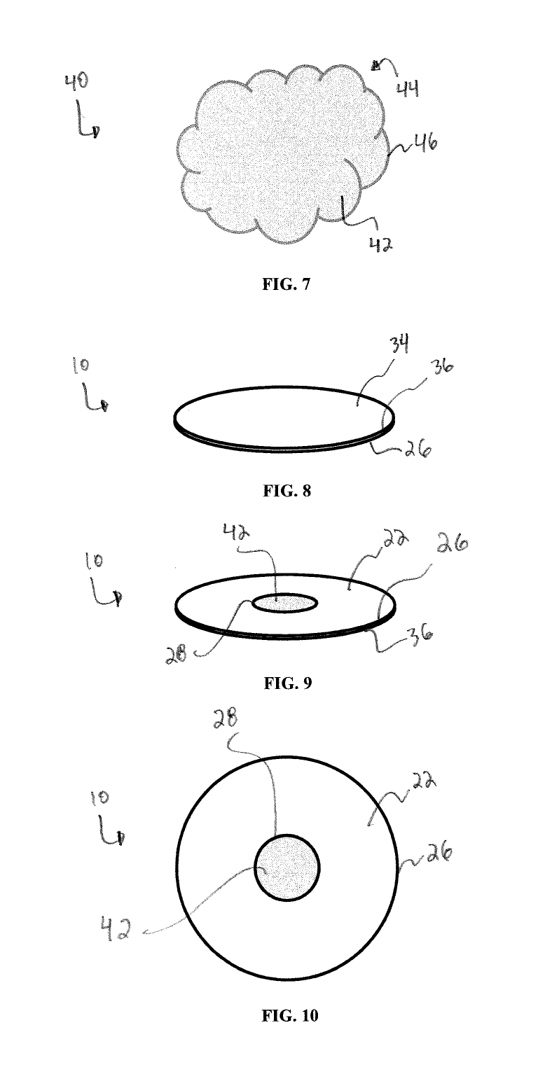

FIGS. 6, 7 and 16 (and elsewhere) show embodiments of the at least one third member 40 comprising at least one third bottom surface 42, at least one third top surface 44, and at least one third perimeter 46. The at least one third perimeter 46 is defined by the entire outer boundary of at least one third member 40. As stated above with regards to each component of device 10, at least one third member 40 and each of its parts may be configured to a variety of positions, sizes, shapes, dimensions, textures, colors, smells, orders, characteristics, and/or designs (including those not illustrated in FIGS. 6, 7 and 16).

In one or more embodiments, at least one third member 40 and each of its parts may be configured from various absorbent material and/or combinations of absorbent materials, including, for example, cotton, cotton balls, hemp, wool, paper, flannel, gauze, bamboo, sponge, foam, micro-fiber, synthetic blend, and the like and/or other materials.

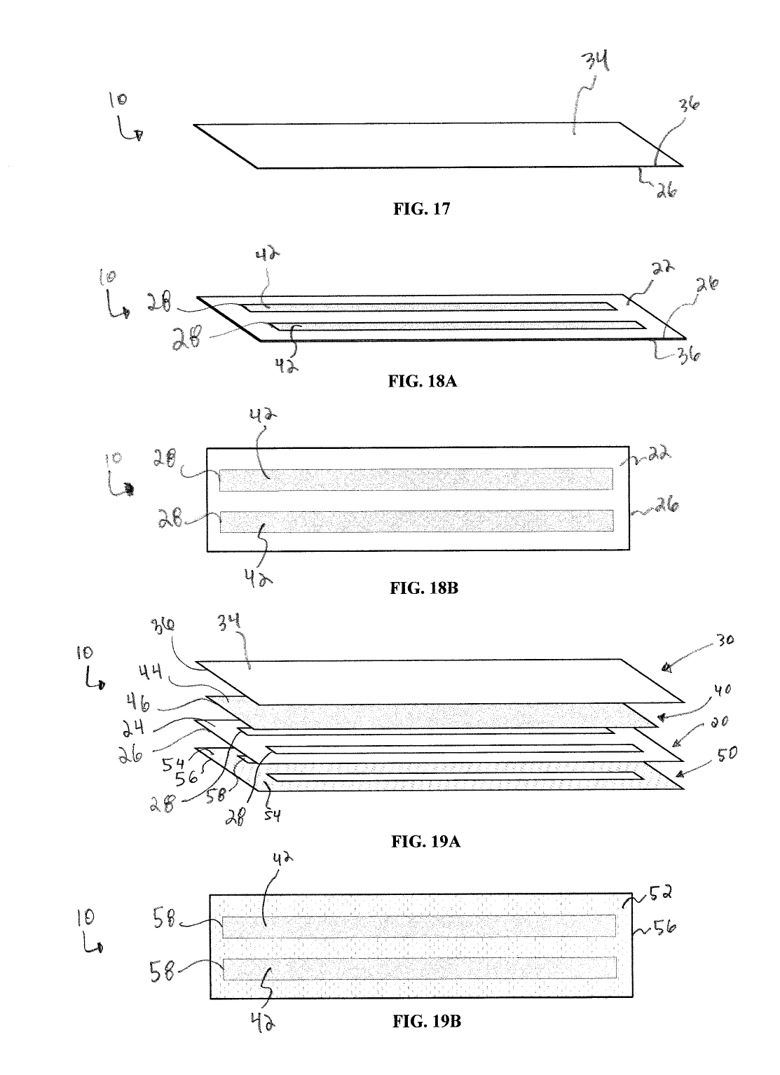

FIGS. 8, 9, 17 and 18A (and elsewhere) show embodiments of device 10 where first perimeter 26 may be configured to second perimeter 36. Such configuration may be accomplished from various materials and/or by various means and/or combinations of the same, including, for example, by gluing, sewing, stapling, folding, pressing, heating, melting, sealing and the like and/or other materials and/or means and/or combinations of the same. The strength of such configuration, in one or more embodiments, may be such that it may be difficult for users to separate first perimeter 26 from second perimeter 36 when removing cover member 50 and/or to ensure that first perimeter 26 remains configured to second perimeter 36 when removing cover member 50 and/or when removing device 10 from a user's skin regardless of whether cover member 50 is included in device 10. Such configuration of first perimeter 26 to second perimeter 36, in one or more embodiments, may be done in a way so as to prevent the seepage of substantially any substances through the points at which first perimeter 26 may be configured to second perimeter 36 and channel the communication of substances through opening 28 and onto a user's skin. In one or more embodiments, the entire or part of first perimeter 26 may be configured to the entire or part of second perimeter 26. In one or more embodiments, first perimeter 26 may be configured to second perimeter 36 in such a way so that at least one third perimeter 46 may be surrounded by said first perimeter 26 and second perimeter 36 and where first member 20 may encapsulate at least one third member 40 on one side (excepting at the at least one opening 28 where at least one third bottom surface 42 may be exposed) and where second member 30 may encapsulate at least one third member 40 on the other side.

In one or more embodiments, at least one third bottom surface 42 may be configured to be near and/or abut first top surface 24; at least one third top surface 44 may be configured to be near and/or abut second bottom surface 32; and/or at least one third member 40 may be configured to fit entirely within first perimeter 26 and second perimeter 36; which configurations may be accomplished by various materials and/or various means and/or combinations of the same.

In one or more embodiments, at least a portion of at least one third member 40 may be accessible through the at least one opening 28. (See FIGS. 1, 9, 10, 11A, 11C, 13, 18A, 18B, 19A and 19B and elsewhere). In one or more embodiments, at least a portion of at least one third bottom surface 42 may be accessible and/or capable of receiving substances through the at least one opening 28.

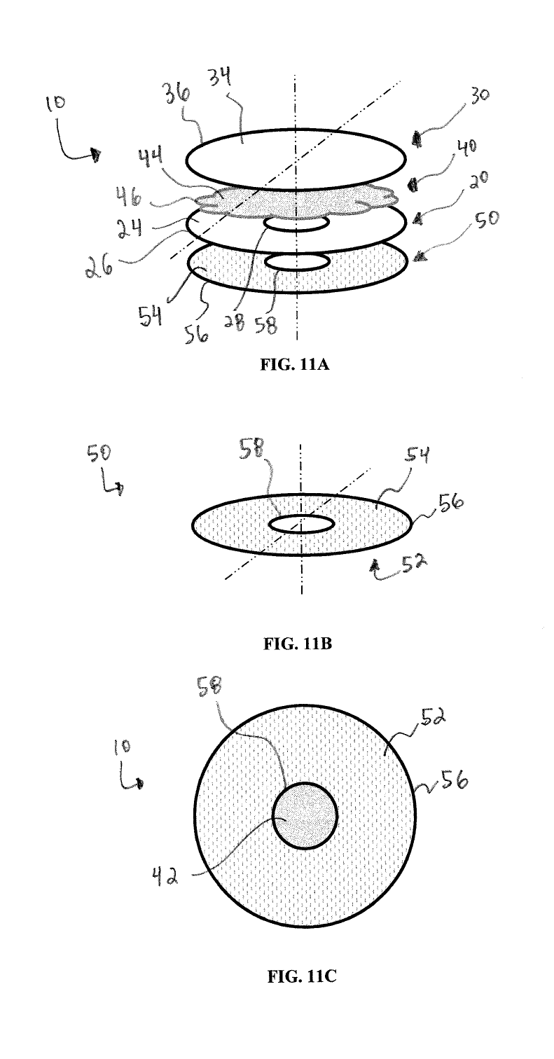

FIGS. 10 and 18B (and elsewhere) show embodiments of device 10 where first member 20 may be configured to surround and/or encapsulate at least one third member 40 excepting at the at least one opening 28 where at least one third bottom surface 42 may be exposed.

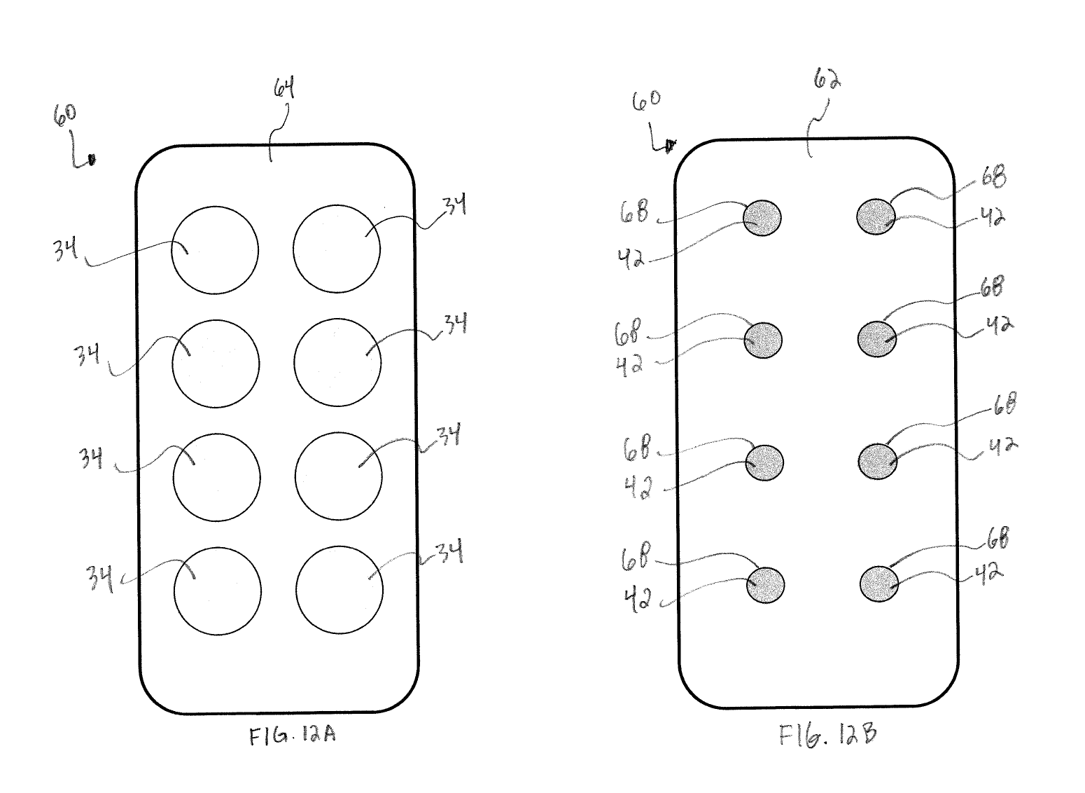

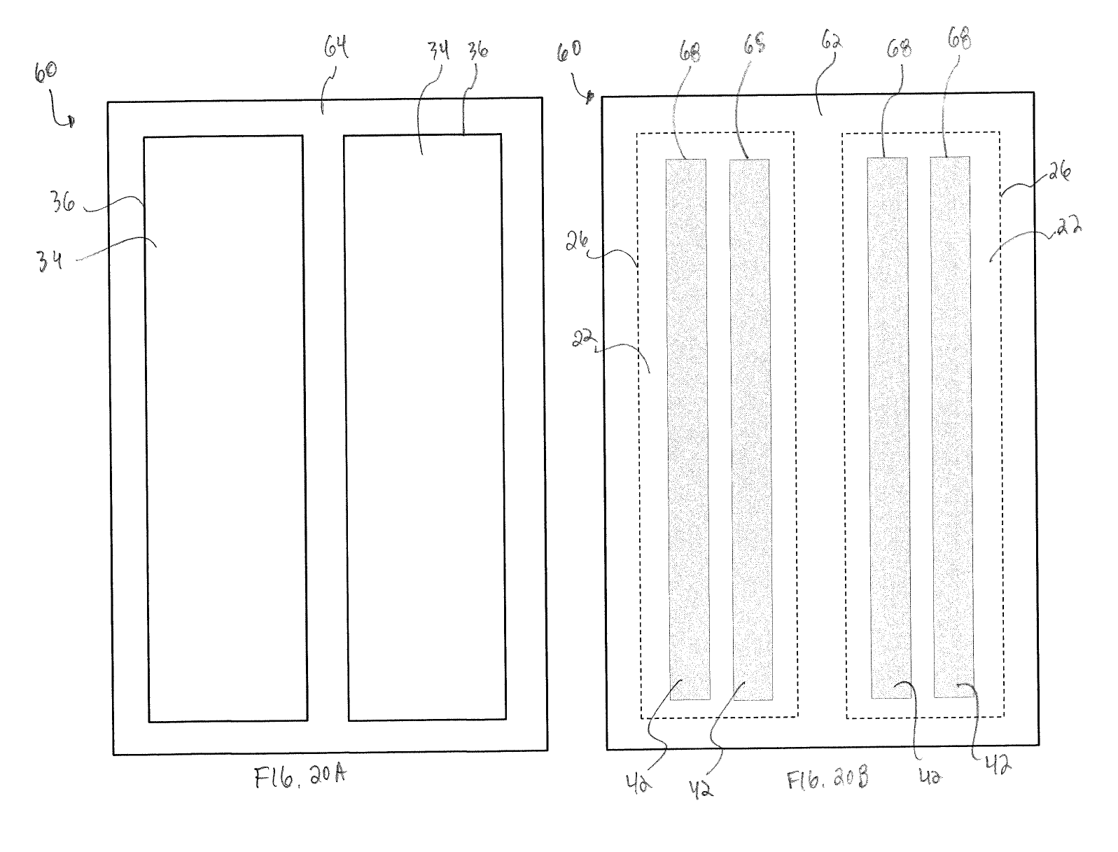

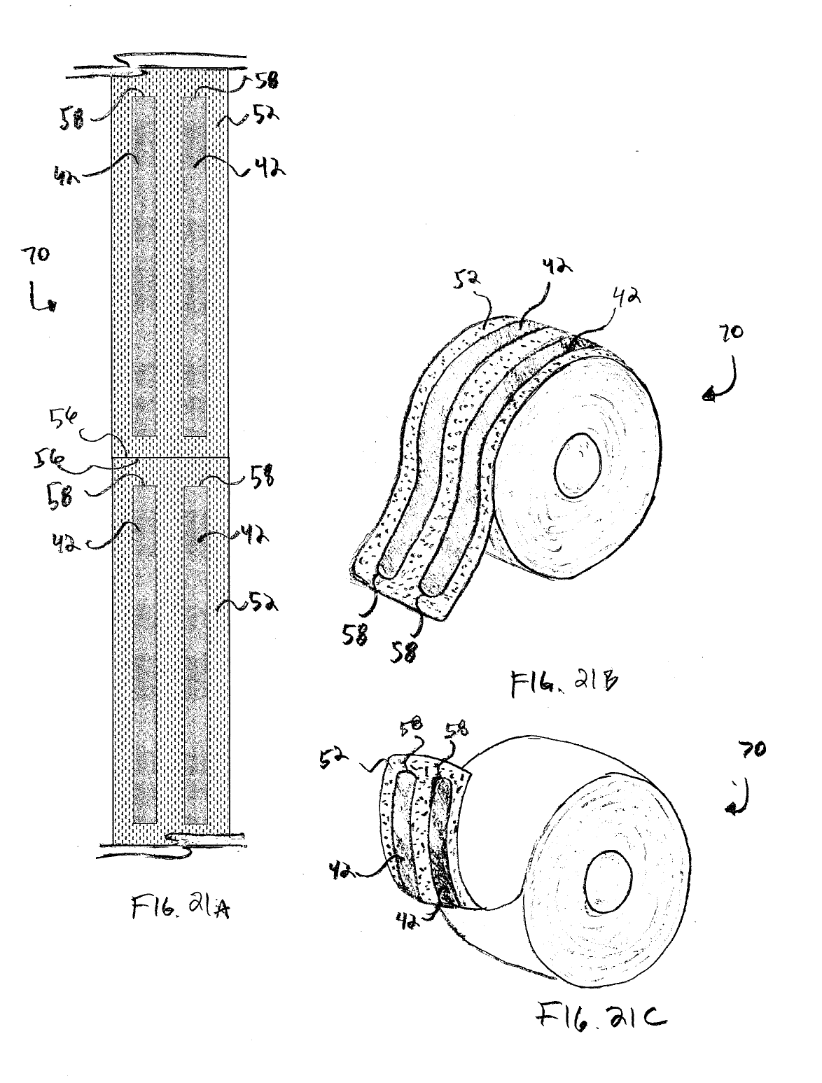

FIGS. 11A and 19A (and elsewhere) show embodiments of device 10 which may include first member 20, second member 30, at least one third member 40, and a cover member 50. In one or more embodiments, cover member 50 may include a cover bottom surface 52, a cover top surface 54, a cover perimeter 56, and at least one cover opening 58. The cover perimeter 56 is defined by the entire outer boundary of cover member 50. The size of the at least one cover opening 58 may vary. As stated above with regards to each component of device 10, cover member 50 and each of its parts may be configured to a variety of positions, sizes, shapes, dimensions, textures, colors, smells, orders, characteristics, and/or designs (including those not illustrated in FIGS. 11A and 19A).

FIG. 11B shows one embodiment of cover member 50 which may include cover bottom surface 52, cover top surface 54, cover perimeter 56 and the at least one cover opening 58.

FIGS. 11C and 19B (and elsewhere) show embodiments of device 10 where cover member 50 may be configured to surround first bottom surface 20 excepting at the at least one cover opening 58. FIGS. 11C and 19B also show embodiments wherein at least a portion of at least one third member 40 (at least one third bottom surface 42 in particular) may be accessible through the at least one cover opening 58. In one or more embodiments, the size of the at least one cover opening 58 may be configured to substantially match or differ from the size of the at least one opening 28 of first member 20. In either case, in one or more embodiments, at least a portion of at least one third bottom surface 42 may be capable of accessing and/or receiving substances through the at least one opening 28 and the at least one cover opening 58.

In one or more embodiments, although not illustrated in FIGS. 11A, 11B, 11C, 19A or 19B, in the instance when first bottom surface 22 is configured from material capable of adhering and/or sticking to a user's skin, at least one purpose of cover member 50 may be to cover, connect to, remain connected to, protect and/or safeguard first bottom surface 22 (and its adhesive material) until a user removes cover member 50. In one or more embodiments, unlike the configuration between first perimeter 26 and second perimeter 36 and although cover member 50 may be connected to first member 20, the connection may be designed to be temporary. In one or more embodiments, cover member 50 may be configured to first member 20 in such ways so as to not undue the configuration of first perimeter 26 to second perimeter 36 when cover member 50 is removed from first bottom surface 22. (It should be apparent to those skilled in the art that referring to removing cover member 50 from first bottom surface 22 (or any other component of device 10) may be interchangeable with referring to removing first bottom surface 22 (or any other component of device 10) from cover member 50).

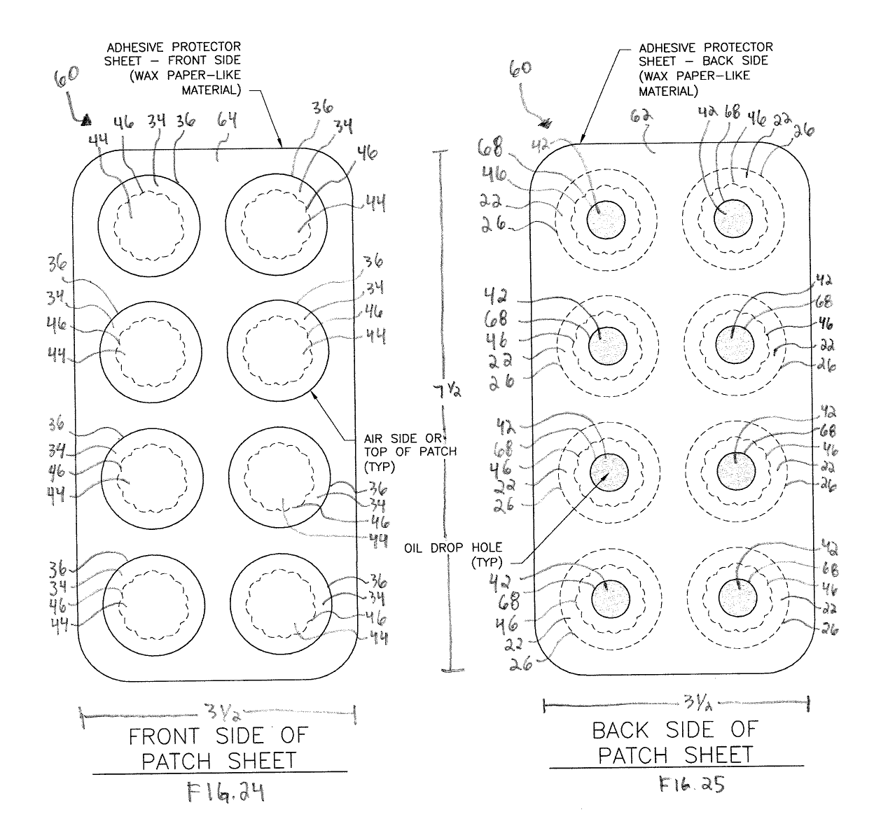

FIG. 21A shows one embodiment of portions of a band 70 which may include at least two devices 10 each of which may include cover bottom surface 52, cover perimeter 56, at least one cover opening 58, at least one third bottom surface 42. FIG. 21A shows one embodiment of portions of a band 70 which may include at least two devices 10, each of which may be substantially as illustrated in FIGS. 19A and 19B, albeit configured together via one cover perimeter 56 configured to another cover perimeter 56. Although FIG. 21A illustrates such a configuration only on certain end of embodied devices 10, it is anticipated that devices 10 may be configured together via any end or border of device 10 by various materials and/or means and/or combinations of the same. For example, two devices 10 may be configured together by perforated ends so that when a user pulls and/or cuts, the devices more naturally pulls apart and/or separates at the perforated configurations. As stated above with regards to device 10, each component of band 70 may be configured to a variety of positions, sizes, shapes, dimensions, textures, colors, smells, orders, characteristics, and/or designs (including those not illustrated in FIG. 21A).

FIG. 21B shows one embodiment of band 70, portions of which may be as illustrated and configured in FIG. 21A, albeit longer and wherein its devices 10 may be rolled up and/or unrolled.

FIG. 21C show the embodiment of band 70 as illustrated and configured in FIG. 21B, albeit with its devices 10 rolled in the opposite direction. Although not illustrated in FIG. 21C, it is anticipated that, in one or more embodiments, this option (as illustrated in FIG. 21C) may remove the necessity of covering member 50 and suggest the use of first member 20, second member 30 and at least one third member 40, along with each of their components. Furthermore, in one or more embodiments, second member 30 may be comprised of various types of medical tapes, wraps, coverings, supports and/or bandages of any kind, such as, for example, kinesiology tape. Thus, not only may a user obtain the benefit of using certain substances (e.g. Essential Oils), but also the benefits associated with whatever medical tape used in conjunction with said substances. Also, when compared to the patch or other like configurations of device 10, bands 70 of devices 10 configured together (including those as illustrated in FIGS. 21A, 21B and 21C, and in FIGS. 13 through 19B) may cover and treat larger target areas.

In one or more embodiments, in addition and/or in alternative to devices 10 being configured as otherwise disclosed herein (such as, for example, as illustrated on in FIGS. 1 through 11C and FIGS. 13 through 19B), multiple devices 10 may be configured together on a sheet or sheets and/or other configurations, by various materials and/or various means and/or combinations of the same. In one or more embodiments, one or more sheets may function similar to sheets of labels, stickers, Band-Aids, stamps or other items which may be removed by users, and which items may possess adhesive qualities, and which items may be placed on and adhere to surfaces. In one or more embodiments, some or all of the components of one or more sheets may be configured from various materials and/or by various means and/or combinations of the same, including, for example, from wax paper, paper, plastic, and other non-stick like material and/or means and/or combinations of the same. As stated above with regards to device 10, each component of one or more sheets may be configured to a variety of positions, sizes, shapes, dimensions, textures, colors, smells, orders, characteristics, and/or designs.

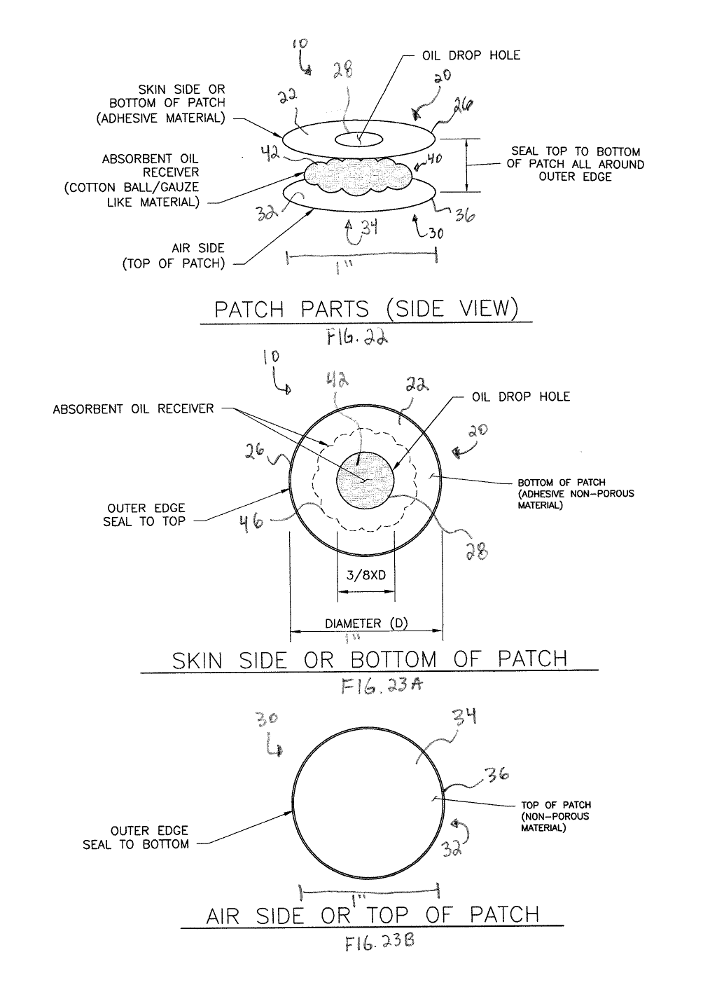

FIGS. 12A, 12B, 20A and 20B (and elsewhere) show embodiments of sheets 60 containing multiple embodiments of device 10.

FIGS. 12A and 20A (and elsewhere) show embodiments of sheets 60 of material including a sheet top surface 64 and with the sheets 60 containing embodiments of multiple devices 10 which include second top surfaces 34 which may substantially face the same direction as sheet top surfaces 64 and substantially the opposite direction as compared to first bottom surfaces 22 (not shown in FIGS. 12A and 20A).

FIGS. 12B and 20B (and elsewhere) show embodiments of the flip side of the sheets 60 of material illustrated in FIGS. 12A and 20A respectively, including a sheet bottom surface 62 and at least one sheet opening 68, as well as including embodiments of multiple devices 10. FIGS. 12B and 20B also show embodiments wherein at least a portion of at least one third member 40 (at least one third bottom surface 42 in particular) may be accessible through the at least one sheet opening 68. In one or more embodiments, the size of the at least one sheet opening 68 may be configured to substantially match or differ from the size of the at least one opening 28. In either case, in one or more embodiments, at least a portion of at least one third bottom surface 42 may be capable of accessing and/or receiving substances through the at least one sheet opening 68 and the at least one opening 28.

Although FIGS. 12A and 12B show embodiments of sheets 60 configured with two columns and four rows of devices 10, the positions, sizes, shapes, dimensions, textures, colors, smells, orders, characteristics, and/or designs of sheet 60 and devices 10 may vary and, consequently, the number of rows and columns may vary, for example, sheet 60 may be configured with eight columns and eleven rows or any number or combination of numbers greater or smaller. Although FIGS. 12A and 12B show one embodiment of sheet 60 configured with similarly sized and shaped devices 10, each and/or any combination of devices 10 on a sheet 60 may differ in positions, sizes, shapes, dimensions, textures, colors, smells, orders, characteristics, and/or designs.

Although FIGS. 20A and 20B show embodiments of sheets 60 configured with two columns and one row of devices 10 positioned vertically, positions, sizes, shapes, dimensions, textures, colors, smells, orders, characteristics, and/or designs of sheet 60 and devices 10 may vary and, consequently, the number of rows and columns may vary, for example, sheet 60 may be configured with devices positioned horizontally with one column and five rows or any number or combination of numbers greater or smaller. Although FIGS. 20A and 20B show one embodiment of sheet 60 configured with similarly sized devices 10, each and/or any combination of devices 10 on sheet 60 may differ in position, size, shape, dimension, texture, color, smell, order, characteristic, and/or design.

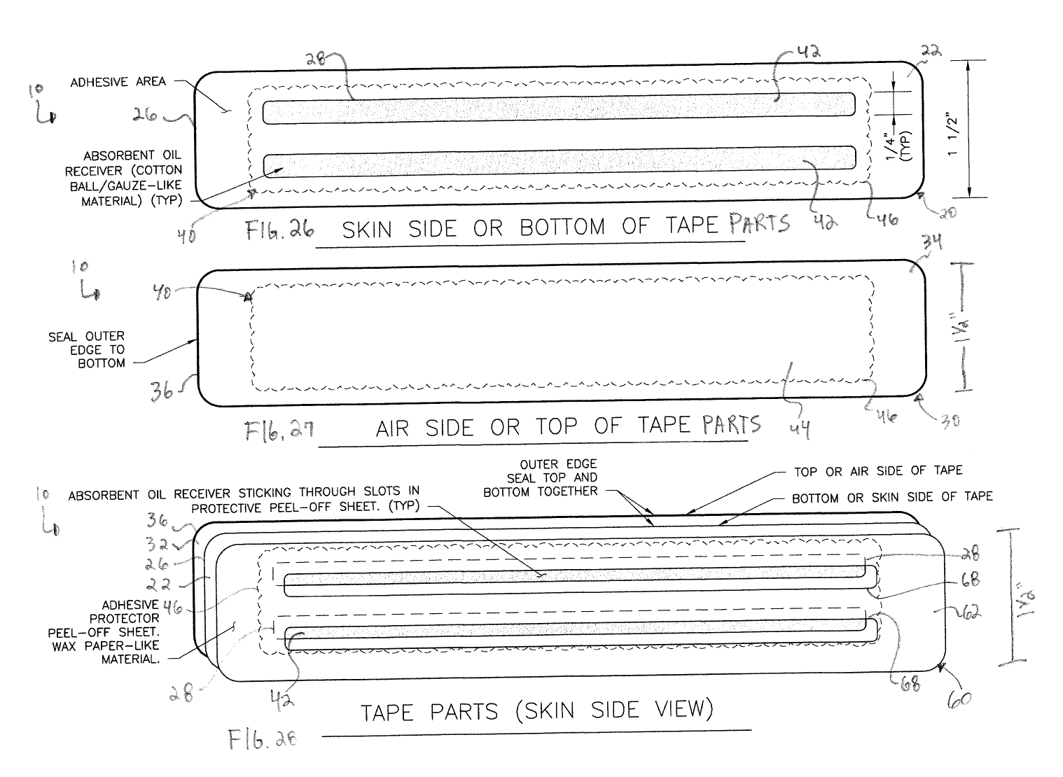

FIGS. 22, 23A and 23B are CAD drawings showing an embodiment of device 10 which may be configured as, like and/or with medical patches.

FIG. 22 is a CAD drawing and shows a perspective view of one embodiment of device 10 with components in a substantially circular shape that may include: first member 20 (i.e. skin side or bottom of patch) which may be between about ⅛ of an inch to about 8 inches in diameter (such as, approximately 1 inch in diameter) and which may be configured to come into contact with a user's skin; first bottom surface 22 (i.e. skin side or bottom of patch or adhesive material) which may be configured from a patch containing adhesive material which may allow device 10 to adhere to and/or stick to a user's skin and/or which may be configured from non-porous material which may prevent and/or channel the seepage of substance from device 10 onto the user's skin; at least one opening 28 (i.e. oil drop hole) through which Essential Oils and/or other substances may be dropped and/or poured which may be between about 1/64 of an inch to about 7 and ¾ inches in diameter (such as, approximately ⅜ of an inch in diameter) (see FIG. 23A); second member 30 (i.e. air side or top of patch) which may be between about ⅛ of an inch to about 8 inches in diameter (such as, approximately 1 inch in diameter) and which may be exposed to the air and/or clothing if device 10 is covered by a user's clothing; second top surface 34 which may be configured from a patch like material (with at least one side optionally configured with non-adhesive material); first perimeter 26 (i.e. bottom side or skin side edge) and second perimeter 36 (i.e. top side or air side edge) which may be sealed and/or configured together around their entire perimeters; and at least one third member 40 (i.e. absorbent oil receiver, cotton ball/gauze like material), the width of which may be less than the diameter of first member 20 and/or second member 30 (such as between about ⅛ of an inch to about 8 inches in diameter) and which may be configured to be a substantially circular and/or cloud like shape, and which may be configured from cotton balls and/or gauze and/or like and/or other materials and which may be configured to receive the oil and/or other substances dropped and/or poured into it by a user. In one or more embodiments, first bottom surface 22 (i.e. skin side of bottom patch) may be configured to completely contain medical skin adhesive to ensure adequate adherence.

FIG. 23A is a CAD drawing and shows a top view of the embodiment of device 10 illustrated in FIG. 22 with components in a substantially circular shape showing first perimeter 26 sealed with second perimeter 36 around there entire perimeters and at least one third perimeter 46 shown in dot line format to signify that at least one third member 40 may be inferior to first member 20. In one or more embodiments, at least one third bottom surface 42 may be configured to protrude slightly out of at least one opening 28. In one or more embodiments, the absorbent material (i.e. at least one third member 40) may be configured to be spread out under bottom patch (i.e. first member 20) to outer edges of patch (i.e. first perimeter 26 and second perimeter 36). In one or more embodiments, the thickness of device 10 may be between about 1/64 of an inch to about ¼ of an inch (such as, approximately 1/16 of an inch) when first perimeter 26 and second perimeter 36 are sealed and/or configured together.

FIG. 23B is a CAD drawing and shows a bottom view of the embodiment of device 10 illustrated in FIG. 22 with second member 30 with in a substantially circular shape and which may include second top surface 34 (i.e. air side of top patch), second perimeter 36 sealed and/or configured together with first perimeter 26 around there entire perimeters and second bottom surface 32 (not shown in FIG. 23B). In one or more embodiments, second top surface 34 may be configured to be printed on with advertising, such as, for example, the logo of seller's company.

FIGS. 24 and 25 are CAD drawings showing the device 10 illustrated in FIGS. 22, 23A and 23B configured as, like and/or with medical patches releasably configured on at least one sheet 60. All of the configurations associated with patch-like devices 10 described, embodied and illustrated in relation to FIGS. 22, 23A and 23B are hereby incorporated into FIGS. 24 and 25.

FIG. 24 is a CAD drawing and shows one embodiment of one side of sheet 60. As shown, sheet 60 may include components that are configured in a substantially rectangular shape. In one or more embodiments, sheet 60 may be designed for at least the purpose of protecting and/or housing one or more devices 10 configured with adhesive material. In one or more embodiments, sheet 60 may be configured from wax paper, paper, plastic, and/or other non-stick like material. In one or more embodiments, one or more devices 10 may be configured to sheet 60 prior to substances (e.g. Essential Oils) being applied, customized and/or dropped onto the devices 10. In one or more embodiments, sheet 60 may be configured to allow devices 10 to be easily removed from sheet 60. In one or more embodiments, sheet 60 may be designed to allow substances (e.g. Essential Oils) that have been loaded into the absorbent material of the devices 10 to remain in said devices 10 after the devices 10 are removed or peeled off of sheet 60 prior to being placed on a user's skin. In one or more embodiments, sheets 60 may be designed for the purpose of allowing adhesive materials which may be configured prior to substances (e.g. Essential Oils) being applied and/or customized and which may allow devices 10 to be removed easily while maintaining substances (e.g. Essential Oils) on absorbent material and to be placed on a user's skin. FIG. 24 shows the front side of sheet 60 (indicated as an adhesive protector on FIG. 24). In one or more embodiments, sheet 60 may be between about 1 inch to about 24 inches in length (such as about 7½ inches in length) and between about 1 inch to about 15 inches in width (such as about 3½ inches in width). In one or more embodiments, sheet 60 may include sheet top surface 64. In one or more embodiments, sheet 60 may protect and/or house one or more devices 10 (such as eight devices 10) wherein said devices 10 may be configured with components in substantially circular shape. In one or more embodiments, sheet 60 may include two columns and four rows. Each device 10 may be configured to be substantially circular in shape and may include: first member 20 (not shown); second member 30 (including second top surface 34 and second perimeter 36); and third member 40 (including at least one third top surface 44 and at least one third perimeter 46) which may be configured from cotton balls and/or gauze and/or like and/or other materials and which may be configured to receive the oil dropped and/or poured into it by a user. As shown in FIG. 24, third member 40 is depicted in a pattern filled darkened color shading and the at least one third perimeter 46 is shown in dot line format to signify that at least one third member 40 may be inferior to second member 30.

FIG. 25 is a CAD drawing and shows one embodiment of the bottom or flip side of the sheet 60 illustrated in FIG. 24. As shown in FIG. 25, sheet 60 may include components designed in a substantially rectangular shape and may include at least one third bottom surface 42 which may be configured to be substantially in circular and/or cloud like shape, and which may be configured from cotton balls and/or gauze and/or like materials, and which may be configured to receive the oil dropped and/or poured into it by a user and which is shown in FIG. 25 part in pattern filled darkened color shading and with the at least one third perimeter 46 shown in dot line format to signify that at least one third member 40 may be inferior to first member 20 and sheet bottom surface 62.

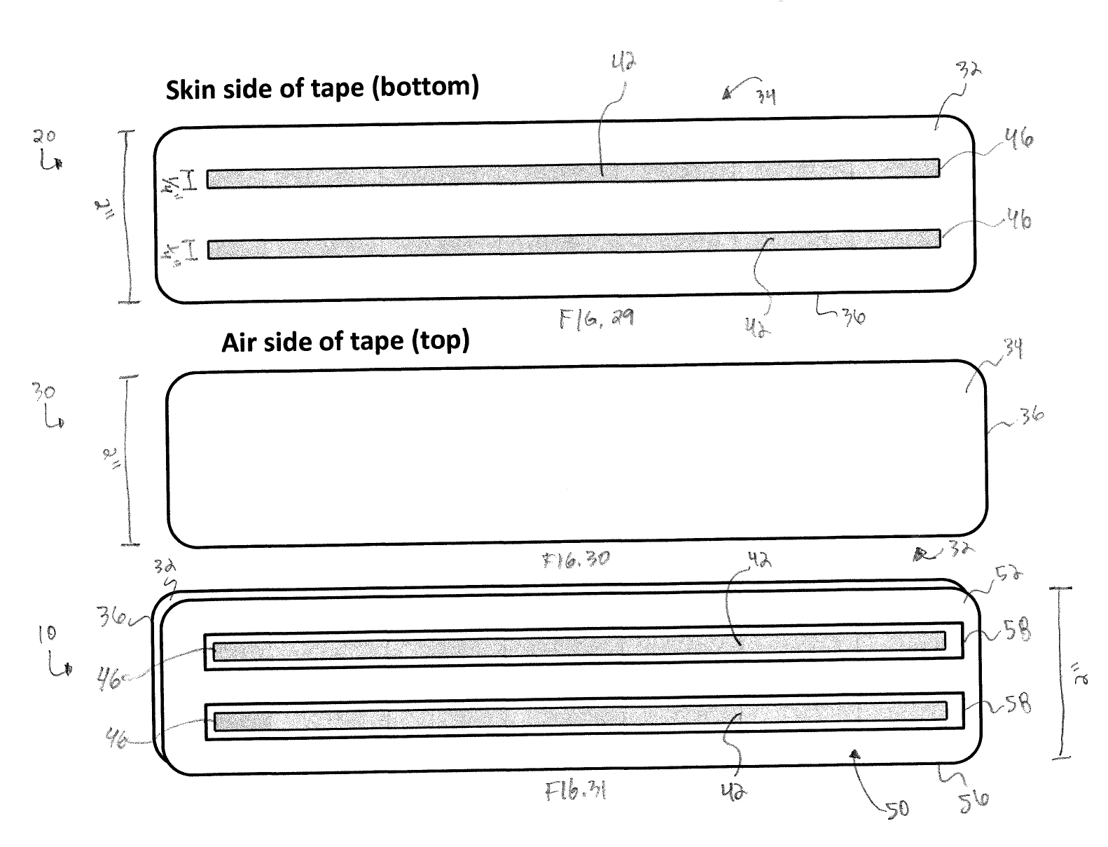

FIGS. 26, 27 and 28 are CAD drawings showing one embodiment of device 10 which may be configured as, like and/or with medical tape, wrapping, covering, support and/or bandaging of any kind, such as, for example, kinesiology tape. Although the length and width of various components described and/or illustrated in FIGS. 26, 27 and 28 may be expressed herein, the width and/or length may be customizable, adaptable and/or varied. In one or more embodiments, device 10 may be designed to be as, on, like and/or with a role of medical tape, wrapping, covering, support and/or bandaging of any kind, such as, for example, kinesiology tape; and, in one or more embodiments, both the length and width of device 10 may be of the length and width in which such products may be used, manufactured and/or customized.

FIG. 26 is a CAD drawing and shows one embodiment of the bottom side of device 10 with components in substantially rectangular shape and which may include: first member 20 which may be between about 1/64 of an inch to about 10 inches in width (such as approximately 1½ inches in width) and between about 1/2 of an inch to about 50 feet in length, noting that the width and length may be customizable and/or adaptable as stated above, and which first member 20 may be configured to come into contact with and/or adhere to a user's skin; first bottom surface 22 which may be configured from adhesive and/or non-porous material; first perimeter 26 and second perimeter 36 (not shown in FIG. 26) which may be sealed together around the entire perimeter of each; at least one opening 28 which may be between about 1/64 of an inch to about 9 and ¾ inches in width (such as approximately ¼ of an inch in width) and between about 1/16 of an inch to about 50 feet in length, noting that the width and length may be customizable and/or adaptable as stated above, and through the at least one opening 28 Essential Oils and/or other substances may be dropped and/or poured and/or otherwise communicated; and at least one third bottom surface 42 which may be configured to be substantially in a rectangular cloud like shape, and which may be configured from cotton balls and/or gauze and/or like materials, and which may be configured to receive the oil and/or other substances dropped and/or poured and/or otherwise communicated into it by a user and which is shown in FIG. 26 in pattern filled darkened color shading and with the at least one third perimeter 46 shown in dot line format to signify that at least one third member 40 may be inferior to first member 20.

FIG. 27 is a CAD drawing and shows one embodiment of device 10 with components in substantially rectangular shape and which may include: second member 30 which may be between about 1/64 of an inch to about 10 inches in width (such as approximately 1½ inches in width) and between about ½ of an inch to about 50 feet in length, noting that the width and length may be customizable and/or adaptable as stated above; second top surface 34; second perimeter 36 and first perimeter 26 (not shown in FIG. 27) which may be sealed together around the entire perimeter of each; at least one third member 40 which may be less than the width and length of second member 30 and first member 20 (such as less than approximately 1½ inches in width), noting that the width and length may be customizable and/or adaptable as stated above; at least one third top surface 44; and at least one third perimeter 46 which may be configured to be substantially in a rectangular cloud like shape, and which may be configured from cotton balls and/or gauze and/or like materials, and which is shown in dot line format to signify that at least one third member 40 may be inferior to second member 30.

FIG. 28 is a CAD drawing and shows one embodiment of sheet 60, with components in substantially rectangular shape. In one or more embodiments, sheet 60 may be designed for at least the purpose of protecting and/or housing one or more devices 10 configured with adhesive material. In one or more embodiments, sheet 60 may be configured from wax paper, paper, plastic, and/or other non-stick like material. In one or more embodiments, one or more devices 10 may be configured to sheet 60 prior to substances (e.g. Essential Oils) being applied, customized and/or dropped onto the devices 10. In one or more embodiments, sheet 60 may be configured to allow devices 10 to be easily removed from sheet 60. In one or more embodiments, sheet 60 may be designed to allow substances (e.g. Essential Oils) that have been loaded into the absorbent material of the devices 10 to remain in said devices 10 after the devices 10 are removed or peeled off of sheet 60 prior to being placed on a user's skin. In one or more embodiments, sheets 60 may be designed for the purpose of allowing adhesive materials which may be configured prior to substances (e.g. Essential Oils) being applied and/or customized and which may allow devices 10 to be removed easily while maintaining substances (e.g. Essential Oils) on absorbent material and to be placed on a user's skin. FIG. 28 shows the bottom side of sheet 60 (indicated as an adhesive protector on FIG. 28). In one or more embodiments, sheet 60 may be between about ¼ of an inch to about 50 inches in length (such as about 7½ inches in length) and between about ¼ of an inch to about 15 inches in width (such as about 1½ inches in width), noting that the width and length may be customizable and/or adaptable as stated above. In one or more embodiments, sheet 60 may include sheet bottom surface 62 and at least one sheet opening 68 which may be configured to receive the oil and/or other substances dropped and/or poured and/or otherwise communicated into it by a user. In one or more embodiments, the at least one sheet opening 68 may be between about 1/64 of an inch to about 9 and ¾ inches in width (such as approximately ¼ of an inch in width) and between about 1/16 of an inch to about 50 feet in length, noting that the width and length may be customizable and/or adaptable as stated above. In one or more embodiments, such as shown in FIG. 28, device 10 may include components in substantially rectangular shape. Device 10 may include first bottom surface 22 which may be configured with adhesive material to come into contact with a user's skin; first perimeter 26 and second perimeter 36 which may be sealed together around the entire perimeter of each; at least one opening 28 shown in dot line format to signify that first member 20 may be inferior to sheet 60; second member 30 which may be exposed to the air and/or clothing if device 10 is covered by a user's clothing; second bottom surface 32; second perimeter 36; at least one third bottom surface 42 which may be configured to be substantially in a rectangular cloud like shape, and which may be configured from cotton balls and/or gauze and/or like materials, and which may be configured to receive the oil dropped and/or poured into it by a user. As shown in FIG. 28 third bottom surface 42 may be partly in pattern filled darkened color shading and with the at least one third perimeter 46 shown in dot line format to signify that at least one third member 40 may be inferior to first member 20 and sheet 60.

FIGS. 29, 30, and 31 shows embodiments of device 10 which may be configured with a second member 30, at least one third member 40, and/or a cover member 50. Second member 30 may include second bottom surface 32, second top surface 34, and second perimeter 36. At least one third member 40 may include at least one third bottom surface 42, at least one third top surface 44, and at least one third perimeter 46. Cover member 50 may include cover bottom surface 52, cover top surface 54, cover perimeter 56, and at least one cover opening 58. Furthermore, FIGS. 29, 30, and 31 shows embodiments of device 10 which may be configured as, like and/or with medical tape, wrapping, covering, support and/or bandaging of any kind, such as, for example, kinesiology tape. Although the length and width of various components described in FIGS. 29, 30, and 31 may be expressed herein, it should be noted that the width and/or length may be customizable and/or adaptable. In one or more embodiments, device 10 may be designed to be as, on, like and/or with a role of medical tape, wrapping, covering, support and/or bandaging of any kind, such as, for example, kinesiology tape, and both the length and width may be of the length and width in which such products may be used, manufactured and/or customized. The embodiments of device 10 shown in FIGS. 29, 30, and 31 may be configured as a band 70 such as, for example, illustrated in FIGS. 21A, 21B, and/or 22B, and/or on a sheet 60 such as, for example, illustrated in FIGS. 20A and/or 20B.

FIG. 29 shows one embodiment of device 10 which may include: second member 30 (i.e. skin or bottom side) which may be configured to be between about 1/64 of an inch to about 10 inches in width (such as approximately 2 inches in width) and between about ½ of an inch to about 50 feet in length, noting that the width and length may be customizable and/or adaptable as stated above; and at least one third member 40 which may be between about 1/64 of an inch to about 10 inches in width (such as approximately ¼ of an inch in width) and between about ½ of an inch to about 50 feet in length, noting that the width and length may be customizable and/or adaptable as stated above. Second member 30 may include: second bottom surface 32 which may be configured with adhesive material such as, for example, kinesiology tape, and which may be configured to adhere to a user's skin; a second top surface 34 which may be configured to face the air or a user's clothing if device 10 is worn under a user's clothing; and a second perimeter 36 which may be defined as the entire outer boundary of second member 30. The at least one third member 40 may include: at least one third bottom surface 42 which may be configured from absorbent material and which may be accessible and/or capable of receiving substances through the at least one cover opening 58 (see FIG. 31); at least one third top surface 44 (not shown); and at least one third perimeter 46 which may be configured to substantially match or differ from the size of the at least one cover opening 58 (see FIG. 31) and in either case, in one or more embodiments, at least a portion of at least one third bottom surface 42 may be capable of accessing and/or receiving substances through the at least one cover opening 58.

FIG. 30 shows one embodiment of second member's 30 air or top side which may include: second bottom surface 32, second top surface 34, and second perimeter 36, which be configured as described in FIG. 29, and, in one or more embodiments, device 10 may be configured to be between about 1/64 of an inch to about 10 inches in width (such as approximately 2 inches in width) and between about ½ of an inch to about 50 feet in length, noting that the width and length may be customizable and/or adaptable as stated above.

FIG. 31 shows one embodiment of device 10 which may include second member 30, at least one third member 40, and a cover member 50. In one or more embodiments, the parts or components of second member 30 and at least one third member 40 may be configured as described in to FIGS. 29 and 30. In one or more embodiments, device 10 may be between about 1/64 of an inch to about 10 inches in width (such as approximately 2 inches in width) and between about ½ of an inch to about 50 feet in length, noting that the width and length may be customizable and/or adaptable as stated above. In one or more embodiments, at least one third member 40 may be configured with the absorbent material (i.e. at least one third member 40) placed on adhesive side (i.e. second bottom side 32) which may be configured down the length of device 10 and/or in some position on device 10 where oils and/or other substances may be added and the adhesive material may be configured to remain in a position to adhere to a user's skin. In one or more embodiments, the thickness of absorbent material (i.e. at least one third member 40) on device 10 may be approximately ⅛ of an inch (noting that each and every instance where thickness is stated herein, such may be customizable and/or adaptable similarly to the width and length as stated above). In one or more embodiments, cover member 50 may include cover bottom surface 52, cover top surface 54, cover perimeter 56, and at least one cover opening 58. The at least one cover opening 58 may be wide enough oils and/or other substances to be communicated to at least a portion of the at least one third member's 40 third bottom surface 42. In one or more embodiments, the at least one cover opening 58 may be between about 1/64 of an inch to about 10 inches in width (such as approximately ¼ of an inch in width) and between about ½ of an inch to about 50 feet in length, noting that the width and length may be customizable and/or adaptable as stated above.

FIGS. 29B, 30B, and 31B show embodiments of device 10 which may be configured with a second member 30, at least one third member 40, and/or a cover member 50. Second member 30 may include second bottom surface 32, second top surface 34, and second perimeter 36. At least one third member 40 may include at least one third bottom surface 42, at least one third top surface 44, and at least one third perimeter 46. Cover member 50 may include cover bottom surface 52, cover top surface 54, cover perimeter 56, and at least one cover opening 58. Furthermore, FIGS. 29B, 30B, and 31B show embodiments of device 10 which may be configured as, like and/or with medical tape, wrapping, covering, support and/or bandaging of any kind, such as, for example, kinesiology tape. Although the length and width of various components described in FIGS. 29B, 30B, and 31B may be expressed herein, it should be noted that the width and/or length may be customizable and/or adaptable. In one or more embodiments, device 10 may be designed to be as, on, like and/or with a role of medical tape, wrapping, covering, support and/or bandaging of any kind, such as, for example, kinesiology tape, and both the length and width may be of the length and width in which such products may be used, manufactured and/or customized. The embodiments of device 10 shown in FIGS. 29B, 30B, and 31B may be configured as a band 70 such as, for example, illustrated in FIGS. 21A, 21B, and/or 22B, and/or on a sheet 60 such as, for example, illustrated in FIGS. 20A and/or 20B.

FIG. 29B shows one embodiment of device 10 which may include: second member 30 (i.e. skin or bottom side) which may be configured to be between about 1/64 of an inch to about 10 inches in width (such as approximately 1 and ½ inches in width) and between about ½ of an inch to about 50 feet in length, noting that the width and length may be customizable and/or adaptable as stated above; and at least one third member 40 which may be between about 1/64 of an inch to about 10 inches in width (such as approximately ¼ of an inch in width) and between about ½ of an inch to about 50 feet in length, noting that the width and length may be customizable and/or adaptable as stated above. Second member 30 may include: second bottom surface 32 which may be configured with adhesive material such as, for example, kinesiology tape, and which may be configured to adhere to a user's skin; a second top surface 34 which may be configured to face the air or a user's clothing if device 10 is worn under a user's clothing; and a second perimeter 36 which may be defined as the entire outer boundary of second member 30. The at least one third member 40 may include: at least one third bottom surface 42 which may be configured from absorbent material and which may be accessible and/or capable of receiving substances through the at least one cover opening 58 (see FIG. 31B); at least one third top surface 44 (not shown); and at least one third perimeter 46 which may be configured to substantially match or differ from the size of the at least one cover opening 58 (see FIG. 31B) and in either case, in one or more embodiments, at least a portion of at least one third bottom surface 42 may be capable of accessing and/or receiving substances through the at least one cover opening 58.

FIG. 30B shows one embodiment of second member's 30 air or top side which may include: second bottom surface 32, second top surface 34, and second perimeter 36, which be configured as described in FIG. 29B, and, in one or more embodiments, device 10 may be configured to be between about 1/64 of an inch to about 10 inches in width (such as approximately 1 and ½ inches in width) and between about ½ of an inch to about 50 feet in length, noting that the width and length may be customizable and/or adaptable as stated above.

FIG. 31B shows one embodiment of device 10 which may include second member 30, at least one third member 40, and a cover member 50. In one or more embodiments, the parts or components of second member 30 and at least one third member 40 may be configured as described in to FIGS. 29B and 30B. In one or more embodiments, device 10 may be between about 1/64 of an inch to about 10 inches in width (such as approximately 1 and ½ inches in width) and between about ½ of an inch to about 50 feet in length, noting that the width and length may be customizable and/or adaptable as stated above. In one or more embodiments, at least one third member 40 may be configured with the absorbent material (i.e. at least one third member 40) placed on adhesive side (i.e. second bottom side 32) which may be configured down the length of device 10 and/or in some position on device 10 where oils and/or other substances may be added and the adhesive material may be configured to remain in a position to adhere to a user's skin. In one or more embodiments, the thickness of absorbent material (i.e. at least one third member 40) on device 10 may be approximately ⅛ of an inch (noting that each and every instance where thickness is stated herein, such may be customizable and/or adaptable similarly to the width and length as stated above). In one or more embodiments, cover member 50 may include cover bottom surface 52, cover top surface 54, cover perimeter 56, and at least one cover opening 58. The at least one cover opening 58 may be wide enough oils and/or other substances to be communicated to at least a portion of the at least one third member's 40 third bottom surface 42. In one or more embodiments, the at least one cover opening 58 may be between about 1/64 of an inch to about 10 inches in width (such as approximately ¼ of an inch in width) and between about ½ of an inch to about 50 feet in length, noting that the width and length may be customizable and/or adaptable as stated above.

The figures disclosed herein may not necessarily be to scale and nor do they necessarily depict the exact positions, sizes, shapes, dimensions, textures, colors, smells, orders, characteristics, and/or designs or any of components of the disclosures (e.g. device 10, sheet 60, band 70, etc.). In one or more embodiments, the components of the disclosures (e.g. device 10, sheet 60, band 70, etc.) may be configured to various positions, sizes, shapes, dimensions, textures, colors, smells, orders, characteristics, and/or designs, from various materials, for various reasons. For example, although FIGS. 1 through 12B and FIGS. 22 through 25 and elsewhere show embodiments of components of device 10 in substantially circular shape and although FIGS. 13 through 21C and FIGS. 26 through 31B and elsewhere show embodiments of components of device 10 in substantially rectangular shape, in one or more embodiments, some or all of the components of device 10 may be configured as substantially a square, oval, oblong, star, heart, etc.

In one or more embodiments, the devices 10, sheets 60, bands 70, the parts thereof, and other disclosures herein, may be configured, used and sold separately and/or in combination.

In one or more embodiments, a user may use a device 10 (which may include any device disclosed and/or anticipated herein, such as, for example, whether it is a device 10 as illustrated in FIGS. 1 through 11C and FIGS. 13 through 19B and elsewhere, and/or from a band 70 of devices 10 such as illustrated in FIGS. 21A through 21C and elsewhere, and/or from a sheet 60 of devices 10 such as illustrated in FIGS. 12A, 12B, 20A and 20B and elsewhere, and/or as otherwise disclosed herein) by using his or her customized amount, type, mixture and level of concentration of the substances desired and pouring and/or dropping said substances through the at least one opening 28 if the device 10 has a first member 20 (and/or the at least one cover opening 58 if the device 10 has a cover member 50 and/or at least one sheet opening 68 if the device 10 is on a sheet 60, and/or onto at least one third bottom surface 42 as the case may be). In one or more embodiments, if the device 10 is configured to be as, on, like and/or with a role of medical tape, wrapping, covering, support and/or bandaging of any kind, such as, for example, kinesiology tape, the user may need to prepare the tape by exposing some or all of the desired length and then apply said substances as stated above. In one or more embodiments, the user may optionally allow the substance sufficient time to be absorbed by the at least one third member 40 and/or other components of the device 10 upon which the substance was applied to.

In one or more embodiments, the user may then remove and/or peel off cover member 50 from first bottom surface 22 and/or second bottom surface 32, as the case may be, if the device 10 has a cover member 50 and/or remove and/or peel the device 10 from sheet top surface 64 of sheet 60 if the device is on a sheet 60, as the case may be.

In one or more embodiments, the user may then place the device 10 containing the substances directly onto his or her or another's skin (via first bottom surface 22 and/or second bottom surface 32, as the case may be, and/or) for the desired length of time. In one or more embodiments, at least a portion of the first bottom surface 22 and/or second bottom surface 32, as the case may be, may be configured from material capable of adhering and/or sticking to a user's skin. In such an instance, the adhesion of first bottom surface 22 and/or second bottom surface 32, as the case may be, to a user's skin may function to hold device 10 in place on the target area once placed thereon. Otherwise, a user may need to use tape, bandages or other securing means to hold device 10 in place on the target area once placed thereon. In one or more embodiments, second top surface 34 may be situated so that it is facing away from the user's skin and towards the air and/or user's clothing if the user is wearing the device 10 under clothing. In one or more embodiments, once device 10 may be placed on the user's skin, at least one third bottom surface 42 may contact the user's skin and/or release the substances poured and/or dropped into it onto the user's skin. In one or more embodiments, the user may remove device 10 when desired. The process may be repeated by a user as often as needed and/or desired.

Any of the structures and functions of any instrument described herein may be combined with any of the structures and function of any device 10, sheet 60 and/or band 70 and/or any of their parts, and/or any other disclosure herein.

Different embodiments of the disclosure may implement the above scenario(s) and/or variations of the above scenario(s). In one or more embodiment, any of the structures, functions, and/or features of any aspect of the disclosure expressly and/or inherently described and/or illustrated herein may be combined with any of the structures, functions, and/or features of any other aspect of the disclosure expressly and/or inherently described and/or illustrated herein. In one or more embodiments, each component of the disclosures may be provided in any color.

In one or more embodiments, other modifications may be made to the embodiments illustrated in the drawings and/or otherwise disclosed herein and/or equivalents, which may include and/or have the capacity to utilize abilities, systems, devices, articles, means, functionality, features, methods and/or uses not expressly and/or impliedly described herein and/or illustrated in the drawings to this application but which may be obvious to one skilled in the art, whether developed later or known at the time of filing.

It should be understood that the present systems, devices, means, methods and structures are not intended to be limited to the particular forms disclosed; rather, they are to cover all combinations, modifications, equivalents and alternatives. A system, device, article, means, method or structure that is configured in a certain way may be configured in at least that way, but may also be configured in ways that are not described or illustrated. The disclosure may be configured to function with a variety of systems, devices, means, methods, and structures. Different materials may be used for individual components. Different materials may be combined in a single component.

The present disclosure may be embodied in other specific forms without departing from its spirit or essential characteristics. It is appreciated that various features of the above described examples and embodiments may be mixed and matched to form a variety of other combinations and alternatives. It is also appreciated that devices, methods and systems disclosed herein should not be limited simply to devices, methods and systems related to applying Essential Oils on human skin. The described embodiments are to be considered in all respects as illustrative and not restrictive. Other embodiments and/or implementations are within the scope of the following claims and at least all changes which come within the meaning and range of equivalency of the claims are to be embraced within their scope. The scope of the disclosure may be indicated by the appended claims rather than by any of the foregoing description.

What is claimed is:

1. A device for applying at least one Essential Oil to a user's skin, comprising:
a first member comprising a first bottom surface, a first top surface, a first perimeter defined by the entire outer boundary of the first member, and at least one opening;
a second member comprising a second bottom surface, a second top surface, and a second perimeter defined by the entire outer boundary of the second member;
at least one third member comprising at least one third bottom surface, at least one third top surface, and at least one third perimeter defined by the entire outer boundary of the at least one third member;
wherein the at least one third member is situated in between the second member and the first member;
wherein a user may access the at least one third member through the at least one opening of the first member;
wherein the at least one third member is made from material that absorbs Essential Oils and communicates the at least one Essential Oil onto a user's skin when at least an aspect of the at least one third member is in contact with a user's skin through the at least one opening of the first member;
wherein the first bottom surface comprising material possessing non-porous qualities in order to aid in the seepage of the at least one Essential Oil onto a user's skin from the at least one third member through the at least one opening of the first member when the at least one Essential Oil is applied to the at least one third member;
wherein the first bottom surface further comprising material possessing adhesive qualities in order to stick to a user's skin;
wherein the second top surface comprising material possessing non-porous qualities in order to prevent seepage of the at least one Essential Oil from the second top surface when the at least one Essential Oil is applied to the at least one third member; and
wherein the first perimeter is operably connected to the second perimeter such that said connection between the first perimeter and the second perimeter substantially remains in place when a user applies the at least one Essential Oil to the device and when the user removes the device from the user's skin.

2. The device of claim 1, wherein one side of the at least one third member is surrounded by the first perimeter and an alternative side of the at least one third member is surrounded by the second perimeter.

3. The device of claim 1, wherein the first member and the second member are each substantially circular in shape.

4. The device of claim 1, wherein multiple devices are releasably and operably connected to a sheet comprising a sheet bottom surface, a sheet top surface and at least one sheet opening, wherein at least a portion of at least one third member may be accessible through the at least one sheet opening such that the at least one Essential Oil may be applied to the devices releasably and operably connected to the sheet.

5. The device of claim 1, wherein the first member and the second member are each substantially rectangular in shape.

6. The device of claim 1, wherein multiple devices are operably connected together.

7. The device of claim 1, wherein multiple devices are operably connected together to form a band wherein multiple devices are operably connected end to end and capable of being rolled together.

8. The device of claim 1, wherein the second member is made from kinesiology tape.

9. The device of claim 1, further comprising:
a cover member comprising a cover bottom surface, a cover top surface, a cover perimeter defined by the entire outer boundary of cover member, and at least one cover opening;
wherein cover member is configured to releasably cover and surround at least some aspect of the first bottom surface for the purpose of covering and protecting at least some aspect of the first bottom surface until a user removes the cover member from the first bottom surface; and
wherein at least a portion of at least one third member may be accessible through the at least one cover opening.

10. A method of using a device for applying at least one Essential Oil to a user's skin, comprising:
applying at least one Essential Oil onto a device comprising:
a first member comprising a first bottom surface, a first top surface, a first perimeter defined by the entire outer boundary of the first member, and at least one opening;
a second member comprising a second bottom surface, a second top surface, and a second perimeter defined by the entire outer boundary of the second member;
at least one third member comprising at least one third bottom surface, at least one third top surface, and at least one third perimeter defined by the entire outer boundary of the at least one third member;
wherein the at least one third member is situated in between the second member and the first member;
wherein a user may access the at least one third member through the at least one opening of the first member;
wherein the at least one third member is made from material that absorbs Essential Oils and communicates the at least one Essential Oil onto a user's skin when at least an aspect of the at least one third member is in contact with a user's skin through the at least one opening of the first member;
wherein the first bottom surface comprising material possessing non-porous qualities in order to aid in the seepage of the at least one Essential Oil onto a user's skin from the at least one third member through the at least one opening of the first member when the at least one Essential Oil is applied to the at least one third member;
wherein the first bottom surface further comprising material possessing adhesive qualities in order to stick to a user's skin;
wherein the second top surface comprising material possessing non-porous qualities in order to prevent seepage of the at least one Essential Oil from the second top surface when the at least one Essential Oil is applied to the at least one third member; and
wherein the first perimeter is operably connected to the second perimeter such that said connection between the first perimeter and the second perimeter substantially remains in place when a user applies the at least one Essential Oil to the device and when the user removes the device from the user's skin;
placing the device containing the at least one Essential Oil directly onto a target area of a user's skin for a desired length of time; and
removing the device after the desired length of time has passed.

11. A method of using a device for applying at least one Essential Oil to a user's skin, comprising:
applying at least one Essential Oil onto the device of claim 2;
placing the device containing the at least one Essential Oil directly onto a target area of a user's skin for a desired length of time; and
removing the device after the desired length of time has passed.

12. A method of using a device for applying at least one Essential Oil to a user's skin, comprising:
applying at least one Essential Oil onto the device of claim 3;
placing the device containing the at least one Essential Oil directly onto a target area of a user's skin for a desired length of time; and
removing the device after the desired length of time has passed.

13. A method of using a device for applying at least one Essential Oil to a user's skin, comprising:
applying at least one Essential Oil onto the device of claim 4;
placing the device containing the at least one Essential Oil directly onto a target area of a user's skin for a desired length of time; and
removing the device after the desired length of time has passed.

14. A method of using a device for applying at least one Essential Oil to a user's skin, comprising:
applying at least one Essential Oil onto the device of claim 5;
placing the device containing the at least one Essential Oil directly onto a target area of a user's skin for a desired length of time; and
removing the device after the desired length of time has passed.

15. A method of using a device for applying at least one Essential Oil to a user's skin, comprising:
applying at least one Essential Oil onto the device of claim 6;
placing the device containing the at least one Essential Oil directly onto a target area of a user's skin for a desired length of time; and
removing the device after the desired length of time has passed.

16. A method of using a device for applying at least one Essential Oil to a user's skin, comprising:
applying at least one Essential Oil onto the device of claim 7;
placing the device containing the at least one Essential Oil directly onto a target area of a user's skin for a desired length of time; and
removing the device after the desired length of time has passed.

17. A method of using a device for applying at least one Essential Oil to a user's skin, comprising:
applying at least one Essential Oil onto the device of claim 8;
placing the device containing the at least one Essential Oil directly onto a target area of a user's skin for a desired length of time; and
removing the device after the desired length of time has passed.

18. A method of using a device for applying at least one Essential Oil to a user's skin, comprising:
applying at least one Essential Oil onto the device of claim 9;
placing the device containing the at least one Essential Oil directly onto a target area of a user's skin for a desired length of time; and
removing the device after the desired length of time has passed.

19. A system comprising:
a device for applying at least one Essential Oil to a user's skin comprising:
a first member comprising a first bottom surface, a first top surface, a first perimeter defined by the entire outer boundary of the first member, and at least one opening;
a second member comprising a second bottom surface, a second top surface, and a second perimeter defined by the entire outer boundary of the second member;
at least one third member comprising at least one third bottom surface, at least one third top surface, and at least one third perimeter defined by the entire outer boundary of the at least one third member;
wherein the at least one third member is situated in between the second member and the first member;
wherein a user may access the at least one third member through the at least one opening of the first member;
wherein the at least one third member is made from material that absorbs Essential Oils and communicates the at least one Essential Oil onto a user's skin when at least an aspect of the at least one third member is in contact with a user's skin through the at least one opening of the first member;
wherein the first bottom surface comprising material possessing non-porous qualities in order to aid in the seepage of the at least one Essential Oil onto a user's skin from the at least one third member through the at least one opening of the first member when the at least one Essential Oil is applied to the at least one third member;
wherein the first bottom surface further comprising material possessing adhesive qualities in order to stick to a user's skin;
wherein the second top surface comprising material possessing non-porous qualities in order to prevent seepage of the at least one Essential Oil from the second top surface when the at least one Essential Oil is applied to the at least one third member; and
wherein the first perimeter is operably connected to the second perimeter such that said connection between the first perimeter and the second perimeter substantially remains in place when a user applies the at least one Essential Oil to the device and when the user removes the device from the user's skin;
at least one Essential Oil; and
at least one user comprising at least one area of skin.
